(19) United States
(12) Patent Application Publication
(10) Pub. No.: US 2023/0130621 A1
(43) Pub. Date: Apr. 27, 2023

(54) APPLICATION OF AGENT IN PREPARATION OF MEDICINE FOR TREATING/INHIBITING PSORIASIS

(71) Applicant: Liangdan Sun, Hefei City (CN)

(72) Inventors: Liangdan Sun, Hefei City (CN); Qi Zhen, Hefei City (CN); Bao Li, Hefei City (CN)

(21) Appl. No.: 17/645,895

(22) Filed: Dec. 23, 2021

(30) Foreign Application Priority Data
Oct. 25, 2021 (CN) 2021112384337

(51) Int. Cl. C07K 16/24; A61P 17/06

(52) U.S. Cl. CPC C07K 16/245; A61P 17/06

(57) ABSTRACT

The invention relates to the field of pharmaceutical technology for treating skin diseases, in particular, to the use of an agent in preparation of a medicine for treating/inhibiting psoriasis. The agent in the invention is aimed at the inhibition or promotion of a relevant signal in dsDNA-AIM2-Caspase1-IL1b signaling pathway, thus realizing the purpose of treating/inhibiting psoriasis. The agent can effectively inhibit the immune response caused by the inflammasome when used in the medicine for treating/inhibiting psoriasis, thus realizing the therapeutic effect on psoriasis and autoimmune diseases. The agent can also promote the inflammasome response, enhance the body's immune response, thus realizing the therapeutic effect on immunodeficiency diseases.

### TECHNICAL FIELD

The invention relates to the field of pharmaceutical technology for treating skin diseases, in particular, to an use/application of an agent in preparation of a medicine for treating/inhibiting (i.e., treating or inhibiting) psoriasis.

### BACKGROUND

Psoriasis, also known as psora, is a common, chronic, and recurrent autoimmune skin disease. The appearances of scaly erythema and plaques in the affected area due to keratinocyte hyperproliferation are the main clinical manifestations of psoriasis. The course of the disease is long, and in some cases, psoriasis would never heal for almost a lifetime of a patient. This disease, commonly occurred in young and middle-aged people, has a relatively great impact on the physical health and mental status of patients. Current studies have shown that cytokine IL17A/F is an important effector in psoriasis. Biological agents targeting IL17A/F have a good therapeutic effect on psoriasis in clinical. However, relevant biological agents are expensive and need to be used continuously in the later period, the cost of treatment is high, and psoriasis cannot be eradicated. The affected area of patients with psoriasis is mainly the skin, and the level of IL17A/F in the circulatory system is not strongly correlated with the disease. Therefore, finding the source of IL17A/F in patients with psoriasis and finding the key factors that target the pathway of IL17A/F are of great significance for the effective treatment of patients with psoriasis.

### SUMMARY OF THE INVENTION

In view of this, the purpose of the invention is to provide the use of an agent in the preparation of a medicine for treating/inhibiting psoriasis. By research on psoriasis pathogenesis, the present inventors have identified the dsDNA-AIM2-Caspase1-IL1b signaling pathway in psoriasis which causes the occurrence or exacerbation of psoriasis. The invention achieves the purpose of treating or inhibiting psoriasis by inhibiting or enhancing various relevant signals in this signaling pathway.

The agent is at least one selected from the following: (1) interleukin 1b (shorted as IL 1b, IL-1b, IL-1B, or IL 1B) antagonists and biological activity inhibitors or functional analogues thereof; (2) dsDNA antagonists and biological activity inhibitors or functional analogues thereof or dsDNA (i.e., double-stranded deoxyribonucleic acid) degrading agents; (3) AIM2 (i.e., absent-in-melanoma-2, or Aim2) antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

Further, the agent is selected from IL-1β monoclonal antibodies.

Further, the dosage form of the medicine for treating/inhibiting psoriasis is a dressing, an oral medicament, a subcutaneous injection, or an intravenous injection.

Further, the invention also provides the use of an agent in the preparation of inhibitors for inhibiting the IL17 secretion by γδ T cells. The IL17 secretion activity of γδ T cells is particularly strong in the epidermis and dermis of patients with psoriasis. Therefore, IL17 secretion by γδ T cells can also be inhibited through the inhibition of the dsDNA-AIM2-Caspase1-IL1b signaling pathway.

The agent is at least one selected from the following: (1) interleukin 1b antagonists and biological activity inhibitors or functional analogues thereof; (2) dsDNA antagonists and biological activity inhibitors or functional analogues thereof or dsDNA degrading agents; (3) AIM2 antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

Further, the purpose of the invention is to provide the use of an agent in the preparation of a medicine for inhibiting the spread of skin tissue lesions. The dsDNA-AIM2-Caspase1-IL1b signaling pathway is also intensely expressed in the activity of skin tissue lesions in vitro.

The agent is at least one selected from the following: (1) interleukin 1b antagonists and biological activity inhibitors or functional analogues thereof; (2) dsDNA antagonists and biological activity inhibitors or functional analogues thereof or dsDNA degrading agents; (3) AIM2 antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

Further, the purpose of the invention is to provide the use of an agent in the preparation of an inhibitor for keratinocyte proliferation in skin tissue, and the agent is at least one selected from the following: (1) interleukin 1b antagonists and biological activity inhibitors or functional analogues thereof; (2) dsDNA antagonists and biological activity inhibitors or functional analogues thereof or dsDNA degrading agents; (3) AIM2 antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

Further, the purpose of the invention is to provide the use of an agent in the preparation of an inhibitor for AIM2 inflammasome response, and the agent is at least one selected from the following: (1) interleukin 1b antagonists and biological activity inhibitors or functional analogues thereof; (2) dsDNA antagonists and biological activity inhibitors or functional analogues thereof or dsDNA degrading agents; (3) AIM2 antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

Further, the purpose of the invention is to provide a method for inhibiting IL17 secretion by γδ T cells in vitro. The method comprises knocking out AIM2-expressing gene and/or IL1b-expressing gene and/or receptor IL1r-expressing in cells ex vivo. The method for knockout can use existing gene editing technology.

Further, the purpose of the invention is to provide a method for inhibiting AIM2 inflammasome response in vitro, wherein the method comprises using at least one of the following agents for interference: (1) interleukin 1b antagonists and biological activity inhibitors or functional analogues thereof; (2) dsDNA antagonists and biological activity inhibitors or functional analogues thereof or dsDNA degrading agents; (3) AIM2 antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

Further, the purpose of the invention is to provide a method for inhibiting keratinocyte proliferation in skin tissue in vitro. The method comprises using at least one of the following agents for interference: (1) interleukin 1b antagonists and biological activity inhibitors or functional analogues thereof; (2) dsDNA antagonists and biological activity inhibitors or functional analogues thereof or dsDNA degrading agents; (3) AIM2 antagonists and biological activity inhibitors or functional analogues thereof; (4) caspase1 antagonists and biological activity inhibitors or functional analogues thereof; (5) interleukin 1b expression inhibitors, dsDNA production inhibitors, AIM2 expression inhibitors or caspase1 expression inhibitors; (6) AIM2 polymerization inhibitors or dsDNA binding agents or functional analogues thereof; (7) Aim2 gene DNA methylation promoting agents; (8) interleukin 1r1 binding agents or interleukin 1r2 binding agents; (9) interleukin 1ra expression promoting agents; (10) interleukin 17a expression promoting agents or interleukin 17f expression promoting agents.

In the invention, the term “comprise” is an open expression, that is, includes the content specified in the invention, but does not exclude other aspects.

In the invention, the term “functional analogue” refers to substances with similar functions, and their structures are not limited to the same, as long as they have the same or similar functions.

In the invention, the term “agonist” refers to a substance or drug that binds to the receptor of a certain biologically active substance and exhibits the effect of the active substance.

In the invention, the term “antagonist” refers to a class of substances that can bind to a receptor but do not have intrinsic activity. Antagonists are divided into competitive antagonists and non-competitive antagonists.

In the invention, the term “expression promoting agent” refers to a substance that can promote the expression of a certain protein or factor.

In the invention, the term “expression inhibitor” refers to a substance that can inhibit the expression of a certain protein or factor.

In the invention, the term “production inhibitor” refers to a substance that can inhibit the production of a certain substance.

In the invention, the term “biological activity inhibitor” refers to a substance capable of inhibiting the biological activity of a certain substance.

The agent provided by the invention can be used in the preparation of a medicine for treating/inhibiting psoriasis and can effectively inhibit the immune response caused by inflammasomes, thus realizing the therapeutic effect on psoriasis and autoimmune diseases. The agent can also promote the inflammasome response, enhance the body's immune response, and thus play a role in the treatment of immunodeficiency diseases.

### BRIEF DESCRIPTION OF DRAWINGS

FIG. 1 shows the expressions of related proteins in the control group and the psoriasis group by immunohistochemistry/fluorescence detection.

FIG. 2 shows the expressions of related proteins in the control group and the psoriasis group by Western.

FIG. 3 shows the detection of IL1b level in a clinical serum sample.

FIG. 4 shows the keratinocyte single cell sequencing of the skin tissue lesion of a patient with psoriasis.

FIGS. 5A and 5B show the nuclear dsDNA content of the skin tissue lesion in normal people and a patient with psoriasis.

FIG. 6 shows the skin of a mouse model of psoriasis induced by imquimod.

FIG. 7 shows the detection of dsDNA-positive cells in the cytoplasm of the skin lesion by the Tunel method.

FIG. 8 shows the cell after different concentrations of dsDNA being transferred into the skin keratinocyte line HaCaT.

FIG. 9 shows the protein expression of downstream signaling pathway at different concentrations of dsDNA by Western.

FIG. 10 shows the protein expression curve of downstream signaling pathway under the same concentration of dsDNA.

FIGS. 11A-11C show the results of human and mouse primary keratinocyte lines and HaCaT tool keratinocytes stimulated with oligodAT and IL17.

FIG. 12 shows the flow cytometry after co-culture of dsDNA in exosomes and PBMC (Peripheral blood mononuclear cell).

FIG. 13 shows the flow cytometry of PI positive cells in normal people and a patient with psoriasis.

FIG. 14 shows the dsDNA content of normal people and a patient with psoriasis.

FIG. 15 shows an image of the relationship between free dsDNA in serum and dsDNA in exosomes.

FIG. 16 shows the flow cytometry after PBMC being transfected with dsDNA (oligo dA-T).

FIG. 17 shows the relative changes of dsDNA in exosomes released by endothelial cells stimulated by cytokines TNFα (tumor necrosis factor-α) and IL17.

FIG. 18 shows the curve over time of dsDNA in exosomes released by endothelial cells stimulated by cytokines TNFα and IL17.

FIG. 19 shows the expression of GSDM family genes in a clinical sample and a psoriasis skin lesion and a normal skin in a mouse model via expression profile.

FIG. 20 shows the experiment of RNAi interference for GSDM.

FIG. 21 shows the effect of different weights of imiquimod on psoriasis in a mouse.

FIG. 22 shows the psoriasis in an IMQ mouse after targeted knockout of the Aim2 and IL1b genes of the Aim2 pathway.

FIG. 23 shows the relationship between the skin lesion area and severity index of psoriasis in an IMQ mouse and time after targeted knockout of the Aim2 and IL1b genes of the Aim2 pathway.

FIG. 24 shows the signal expression of the aim2 pathway in an IMQ mouse after targeted knockout of the Aim2 and IL1b genes of the Aim2 pathway.

FIG. 25 shows the psoriasis of an IMQ mouse-oligo dAT after targeted knockout of the Aim2 and IL1b genes of the Aim2 pathway.

FIG. 26 shows the spleen after injection of IL-1b into the ears of mice.

FIG. 27 shows the psoriasis skin lesion area and severity index after injection of IL-1b into the ears of mice.

FIG. 28 shows the phenotype of an imq mouse model after the use of IL-1b antibody.

FIGS. 29A and 29B show the phenotype of a mouse induced by imiquimod after KO of the gene of IL1b receptor IL1r.

FIG. 30 shows the flow cytometry of IL17 secretion by γδ T cells in an induced mouse model with psoriasis.

FIG. 31 shows the fluorescence detection of IL17 secretion by γδ T cells in different mouse models.

FIG. 32 shows the flow cytometry of IL17 secretion by γδ T cells in different mouse models.

FIG. 33 shows the flow cytometry of IL17 secretion by γδ T cells in different mouse models.

FIG. 34 shows a schematic diagram of the in vitro stimulation process after co-culture of mouse keratinocytes and immune cells.

FIG. 35 shows the content of IL17 in mouse supernatant after in vitro stimulation.

FIG. 36 shows the immunofluorescence of keratinocytes.

FIG. 37 shows the signal expression of AIM2 pathway in different mouse models.

FIGS. 38A and 38B show the psoriasis skin lesion area and severity index of a clinical mouse skin sample.

FIG. 39 shows the flow cytometry of keratinocytes and IL1B expression.

FIG. 40 shows the results of flow cytometry of immune cells in the epidermis and dermis.

FIG. 41 shows the immunofluorescence detection of immune cells in the epidermis and dermis.

FIG. 42 shows the expression of IL17 in immune cells in the epidermis and dermis.

FIG. 43 shows the transcriptome sequencing.

FIG. 44 shows the detection of DNA methylation level.

FIG. 45 shows the ATAC sequencing.

FIG. 46 shows the effect of AIM2 E32K mutant on psoriasis.

FIG. 47 shows a verification about mutation of 32AA from an acidic amino acid to a basic amino acid affecting the polymerization ability of AIM2.

### DETAILED DESCRIPTION OF EMBODIMENTS

The examples given are to better illustrate the invention, but the content of the invention is not limited to the examples given. Therefore, the non-essential improvements and adjustments made by those skilled in the art to the examples based on the above-mentioned content of the invention still belong to the protection scope of the invention.

### Example 1 Verification of dsDNA-AIM2-Caspase1-IL1b Pathway Activation in Skin Lesions of Patients with Psoriasis

Our previous research found that the free dsDNA in the circulatory system and skin of a patient with psoriasis increased significantly. In order to detect the effect of this change on the body, this example of the invention tested its downstream receptor AIM2 and effector pathways, and the results are shown in Table 1 below. The RNA-seq results of the inventors and the European population both showed that the dsDNA-AIM2-Caspase1-IL1b pathway was activated.

TABLE 1
dsDNA-AIM2-Caspase1-IL1b pathway related protein expression

| Gene | N2C | P2N | P2C | RankP | aseMedia | ontMedia | FC |
|---|---|---|---|---|---|---|---|
| AIM2 | 1 | 5.00E−05 | 5.00E−05 | 2.05E−29 | 1.29 | 0.20 | 6.41 |
| CASP1 | 0.46805 | 0.001 | 0.06295 | 3.96E−18 | 28.87 | 19.57 | 1.48 |
| CASP4 | 0.248 | 5.00E−05 | 5.00E−05 | 3.58E−28 | 56.22 | 30.18 | 1.86 |
| CASP5 | 0.248 | 5.00E−05 | 5.00E−05 | 3.79E−26 | 0.70 | 0.07 | 9.82 |
| IL1b | 0.71135 | 5.00E−05 | 5.00E−05 | 1.06E−22 | 2.82 | 0.42 | 6.77 |
| PYCRAD | 0.8407 | 5.00E−05 | 5.00E−05 | 9.72E−22 | 100.89 | 50.21 | 2.01 |
| MLKL | 0.2779 | 5.00E−05 | 5.00E−05 | 2.70E−23 | 4.83 | 2.39 | 2.02 |

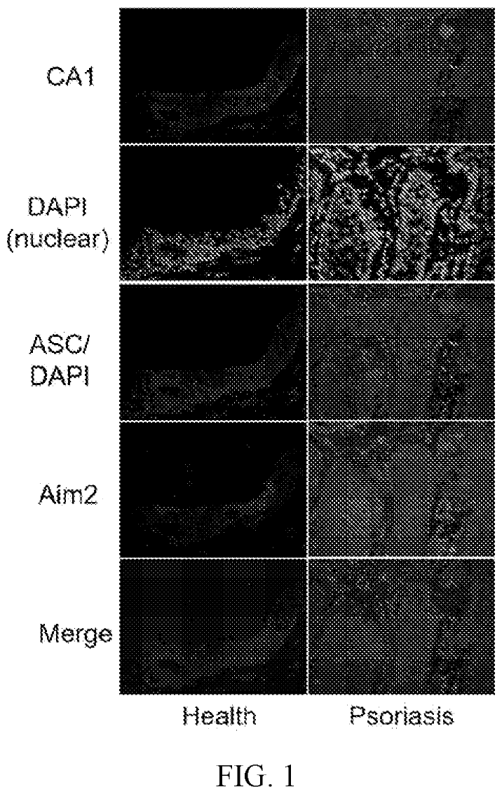

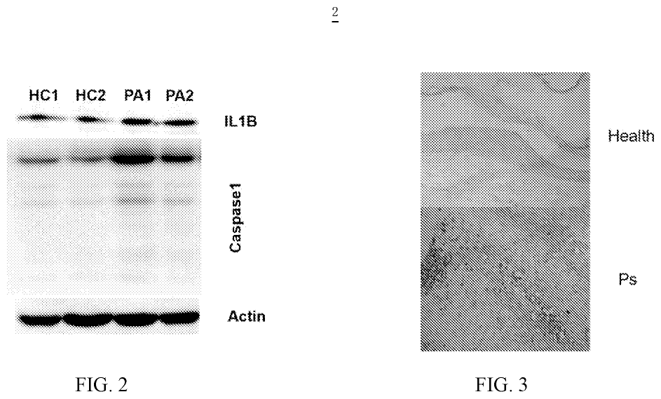

The protein levels were detected by Western and immunohistochemistry/fluorescence. The results are shown in FIG. 1 and FIG. 2. It can be seen that the protein expression of AIM2-Caspase1-IL1b pathway in psoriasis lesions is up-regulated.

The IL1b level in a clinical serum sample was detected, the level of IL1b in the serum of a patient with psoriasis and normal people was compared. The results in FIG. 3 show that the level of IL1b in the patient with psoriasis is significantly higher than that in normal people.

### Example 2 Verification by Single-Cell Sequencing

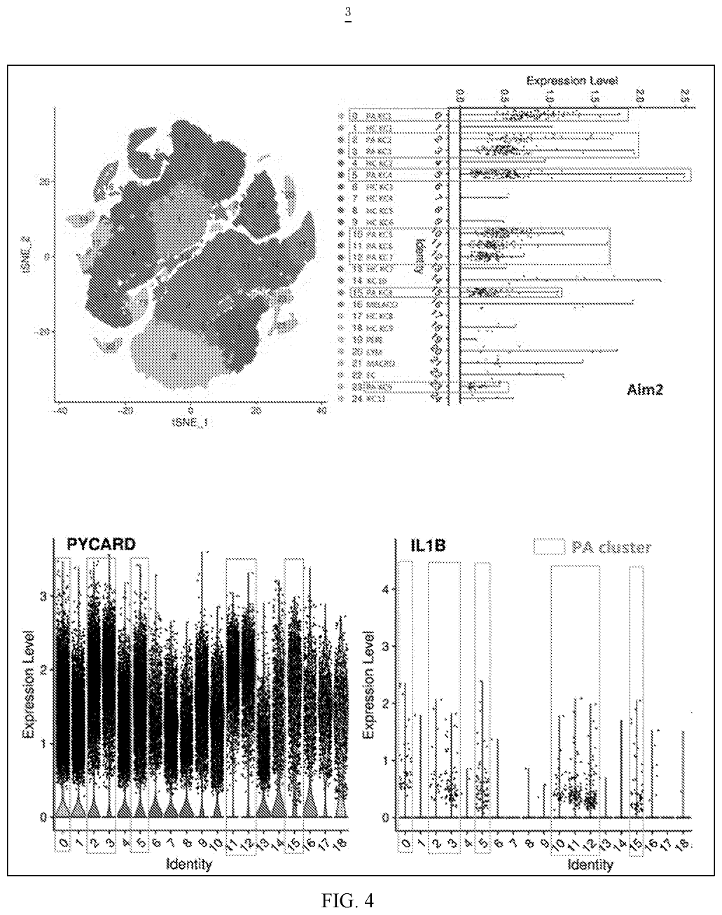

Single-cell sequencing of keratinocytes in the skin lesions of patients with psoriasis was performed. The results in FIG. 4 show that the AIM2 pathway of multiple populations of keratinocytes in the skin lesions of the patients with psoriasis are specifically activated, while the AIM2 pathway in normal human skin is in an inactive state.

### Example 3 Verification by Nuclear dsDNA Detection in Skin Lesions

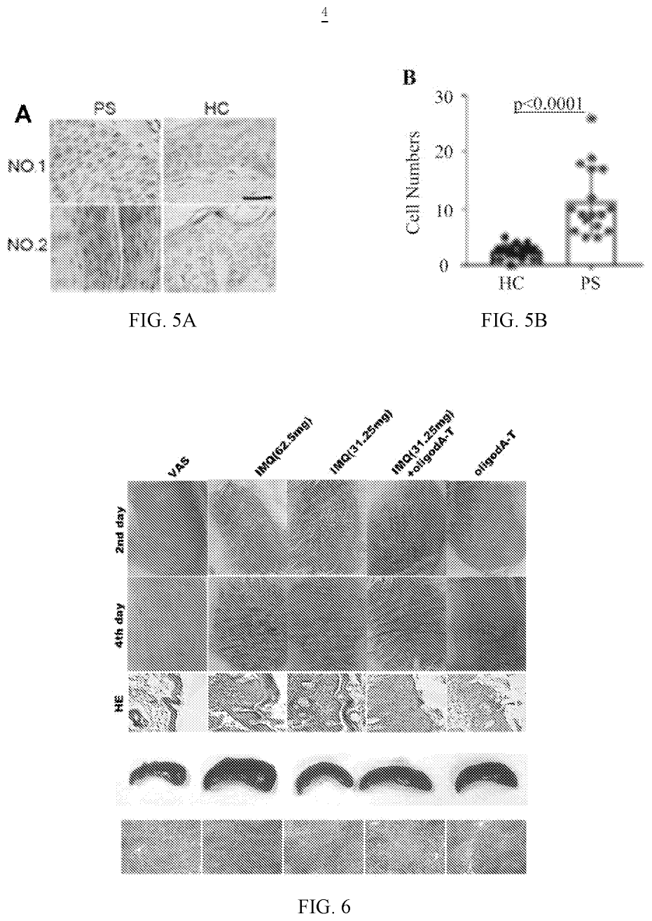

The nuclear dsDNA in the skin lesions of a patient with psoriasis and normal people was detected. The results are shown in FIGS. 5A and 5B. The nuclear dsDNA content in the skin lesions in the patient with psoriasis is higher than the nuclear dsDNA content in normal skin.

### Example 4 Verification by Skin Tissue Detection

In order to verify the experimental results of clinical samples, this example of the invention used the classic imquimod-induced psoriasis mouse model (hereinafter referred to as imi mouse) to detect its skin tissue, and the detection is shown in FIG. 6.

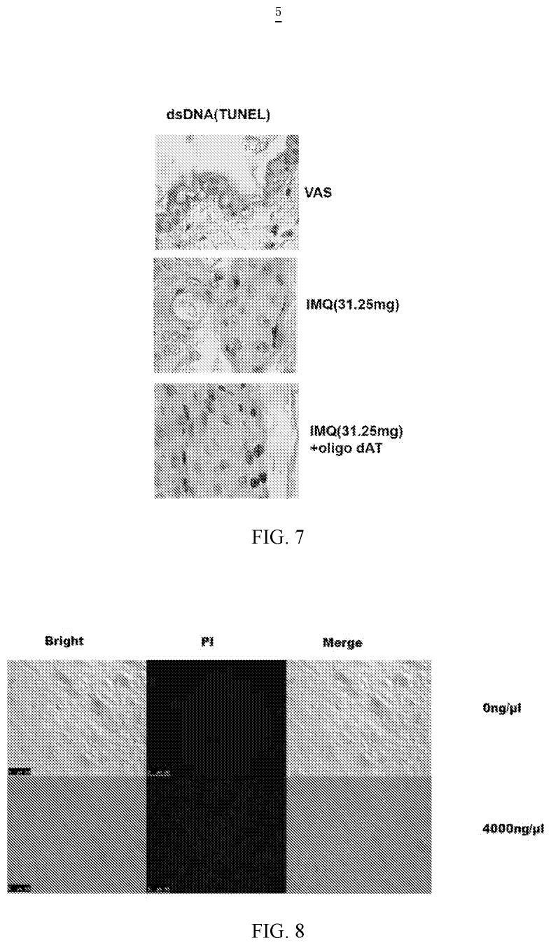

The number of dsDNA positive cells in the cytoplasm in the skin lesions was found to be significantly increased by the Tunel method (the results are shown in FIG. 7), showing that the expression of dsDNA-AIM2-Caspase1-IL1b pathway in the psoriasis mouse model was increased, and the severity of skin lesions was positively correlated with the protein expression of the pathway.

### Example 5 Verification of the Effect of Free dsDNA in Keratinocytes on Cells

In the clinical sample detection in Example 4, it was found that the free dsDNA content in the keratinocytes in the skin lesions of a patient with psoriasis was significantly higher than that of the control skin.

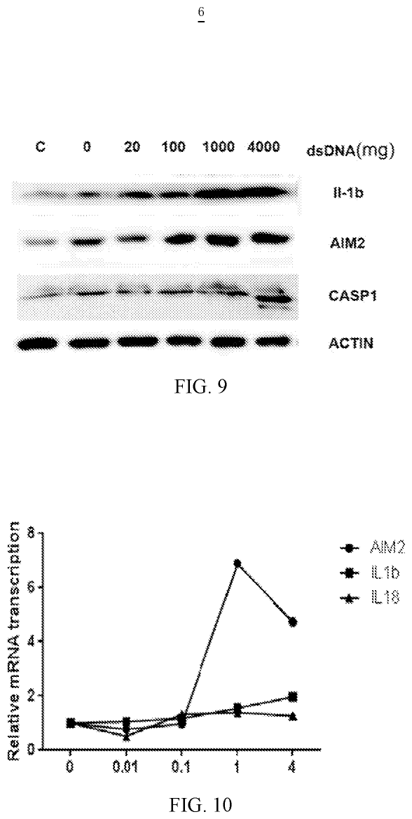

dsDNA can activate the dsDNA-Aim2 pathway in keratinocytes. In order to detect the effect of free dsDNA in keratinocytes on cells, this example of the invention used classic lipfectine3000 to package different concentrations of dsDNA, the packaged dsDNA was transferred into the skin keratinocyte cell line HaCaT, and then the signal expression in downstream pathways of dsDNA was detected. The results in FIG. 8, FIG. 9 and FIG. 10 show that dsDNA can activate the dsDNA-Aim2 pathway and induce keratinocyte pyroptosis, mainly releasing the cytokine IL1b. Lipofectamine3000 was diluted with DMEM and blended thoroughly. Oligodat was diluted with DMEM and P3000 was added and blended thoroughly. The prepared lipofectamine3000 was added to the prepared equal volume of P3000, blended thoroughly, and incubated at room temperature for 5 minutes. The prepared transfection reagent was added to the cell culture medium as needed.

### Example 6 Verification of the Relationship Between AIM2 Pathway and IL17

IL17 aggravates keratinocyte pyroptosis and IL1b release induced by dsDNA. IL17 is an important clinically relevant cytokine for psoriasis. The inventors found that the dsDNA-AIM2-IL1B pathway was activated in clinical samples, animal models, and keratinocyte models.

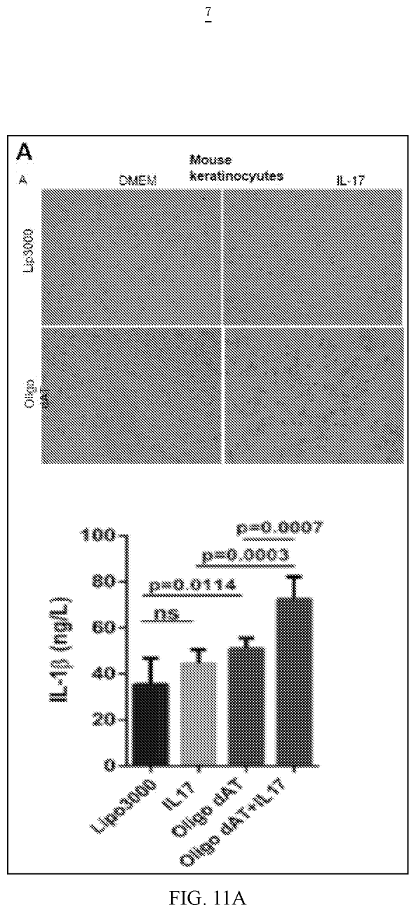

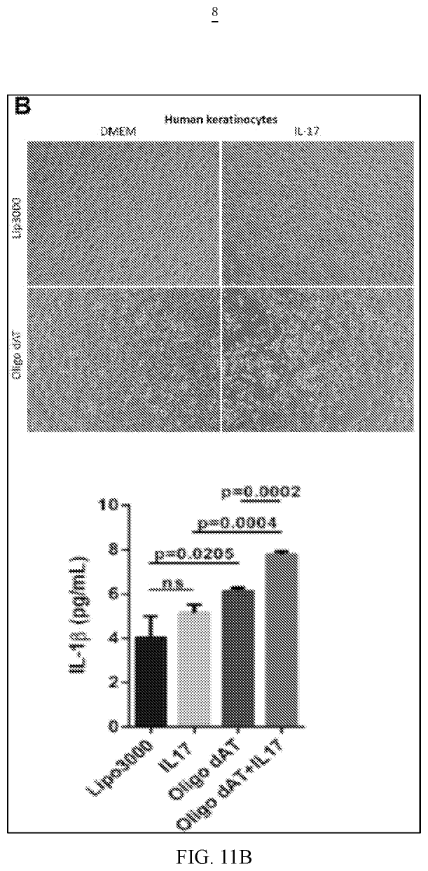

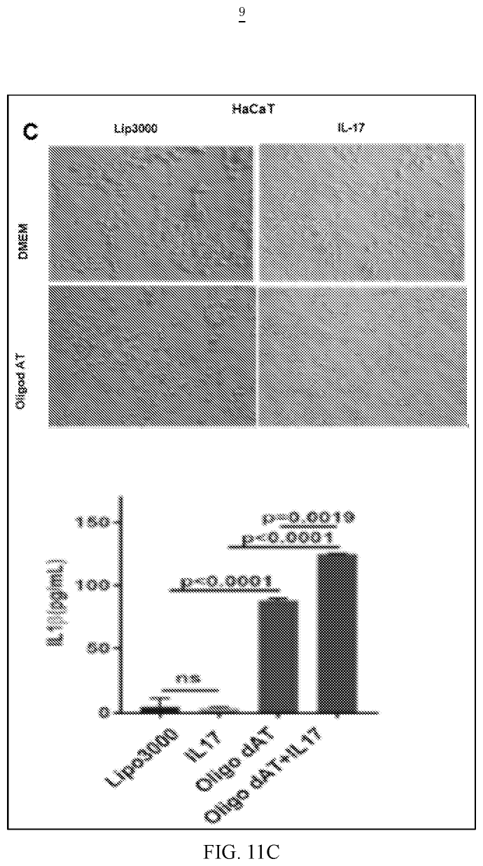

In order to identify the relationship between the AIM2 pathway and IL17, this example of the invention used human and mouse primary keratinocyte lines and HaCaT tool keratinocyte lines stimulated with oligodAT, IL17, a combination of oligodAT and IL17, respectively. The stimulation results are shown in FIGS. 11A-11C. The results show that IL17 can significantly enhance and amplify the stimulating effect of oligodAT, including the occurrence of pyroptosis and the release of IL1b. The results further confirm the important role of the dsDNA-AIM2 pathway in the pathogenesis and exacerbation of psoriasis.

### Example 7

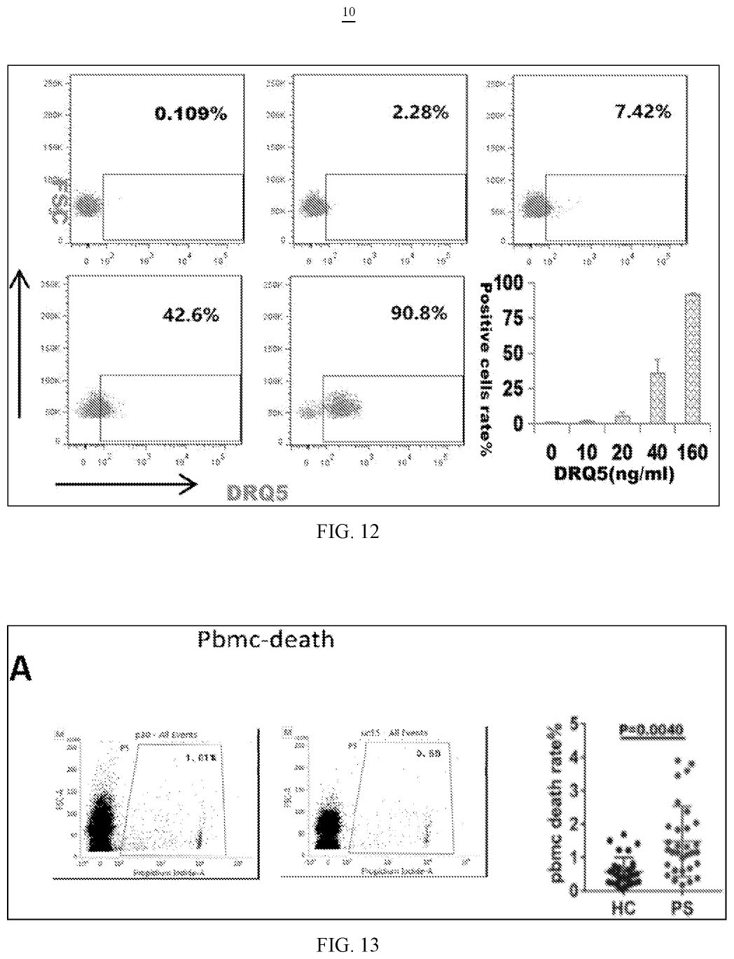

In this example of the invention, the dsDNA in the isolated exosomes was labeled with DRQ5, and then co-cultured with PBMC. The culture results are shown in FIG. 12, and the results show that the exosomes can effectively bring dsDNA in the body into target cells.

### Example 8

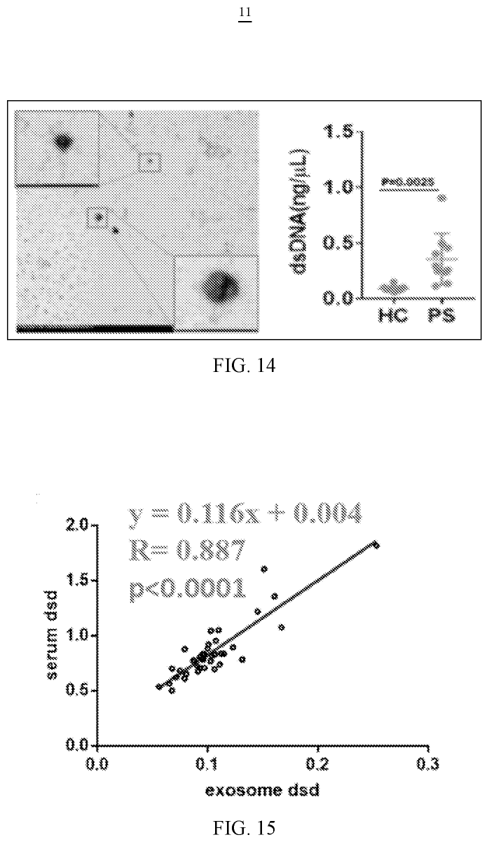

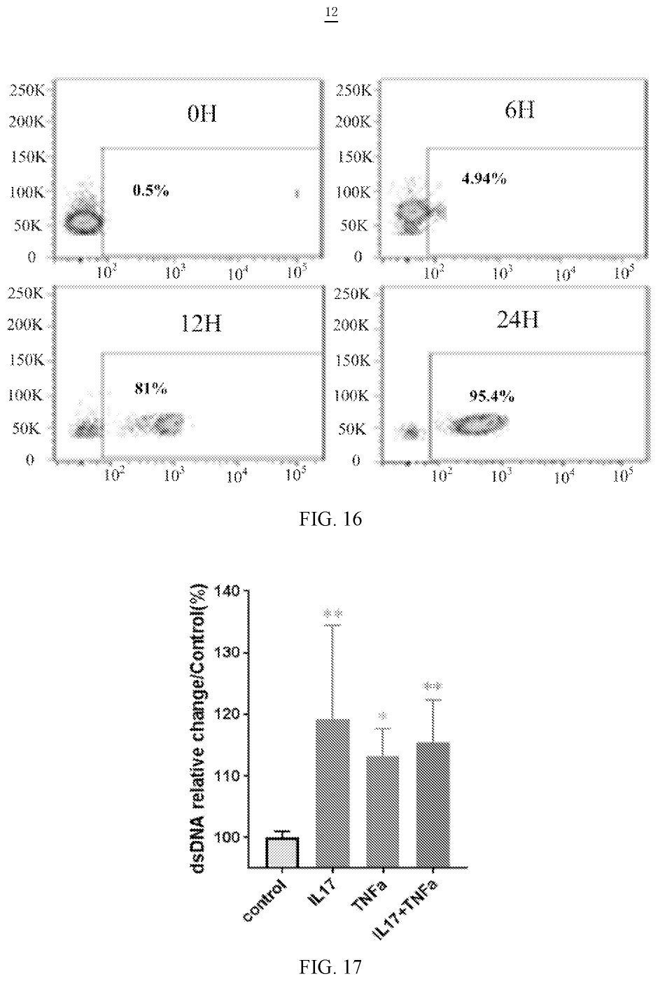

Both apoptosis and necrosis of PBMC release dsDNA. Therefore, this example of the invention used flow cytometry to detect the death of PBMC in normal people and a patient with psoriasis. The results show that the PI-positive cells of PBMC in the patient with psoriasis are significantly higher than those in normal people (as shown in FIG. 13); FIG. 14 shows that the isolation and identification of serum exosomes showed that the dsDNA content in a patient was significantly higher than that in normal people and the free dsDNA in serum and the content of dsDNA in exosomes were significantly linearly correlated (as shown in FIG. 15). Then dsDNA (oligo dA-T) was used to transfect PBMC. The results in FIG. 16 show that dsDNA entering the cytoplasm can induce aggravation of psoriasis.

### Example 9

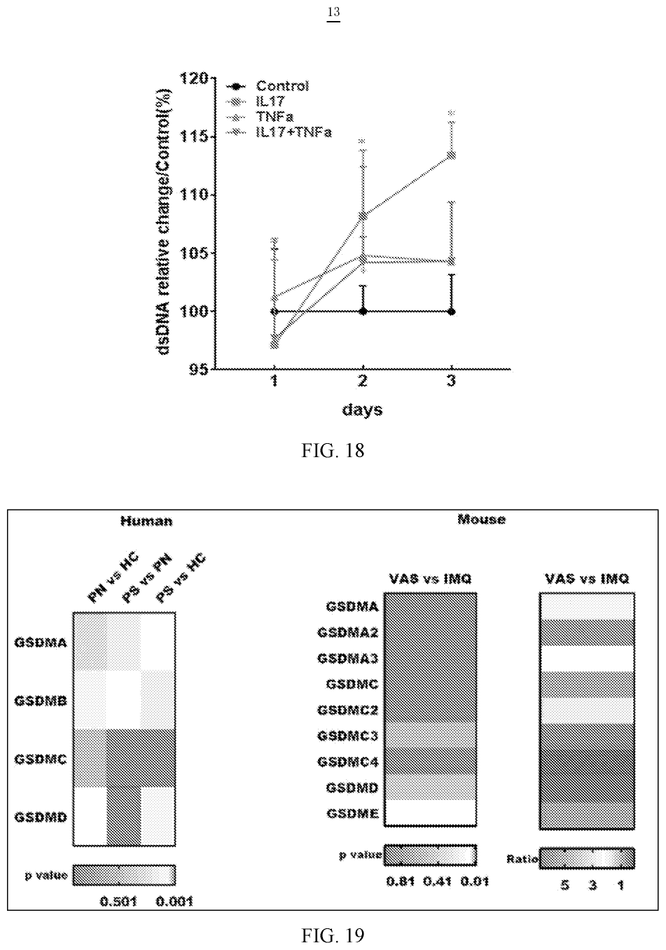

The dsDNA in exosomes released by endothelial cells stimulated by cytokines TNFα and IL17 was detected. The results are shown in FIG. 17 and FIG. 18. The cytokines TNFα and IL17 significantly stimulated the dsDNA content in exosomes released by endothelial cells.

### Example 10

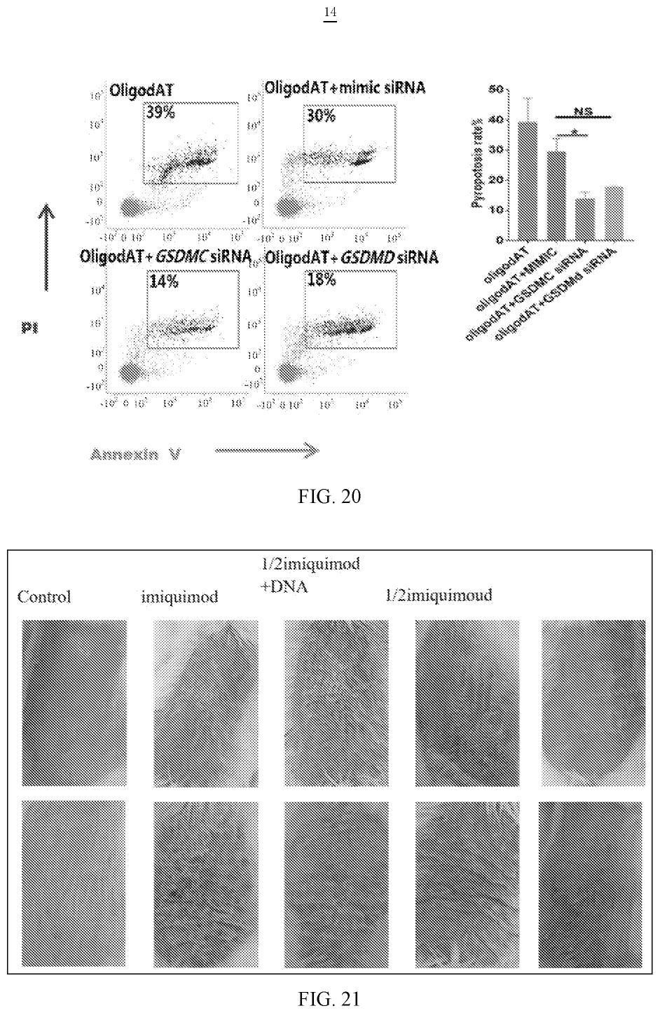

This example of the invention used expression profile to detect the expression of GSDM family genes in a clinical sample and a psoriasis skin lesion and a normal skin in a mouse model. The results are shown in FIG. 19. The RNAi interference experiment for GSDM was performed. The results in FIG. 20 show that gsdmc plays a more important role in the activation of the Aim2 pathway in HaCaT cells.

### Example 11

In this example of the invention, a dsDNA (in vivo/in vitro administration) aggravation experiment was performed. The results are shown in FIG. 21. Aggravated and prolonged psoriasis phenotype is observed in imq model+lip− (oligo dat), and the psoriasis response is the most obvious when using lip to package dsDNA to deliver the dsDNA into cells. (PASI and HE/tunel: dsDNA A. in cytoplasma B. higher level in ps skin lesion/western: aim2 pycard caspase1) The detection of pathway-related genes indicated that the pathway was activated.

### Example 12

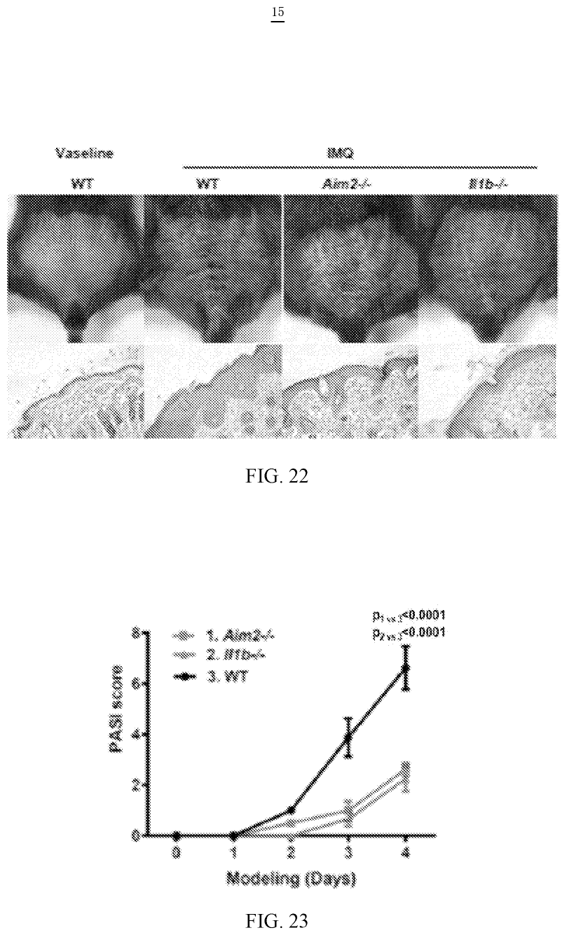

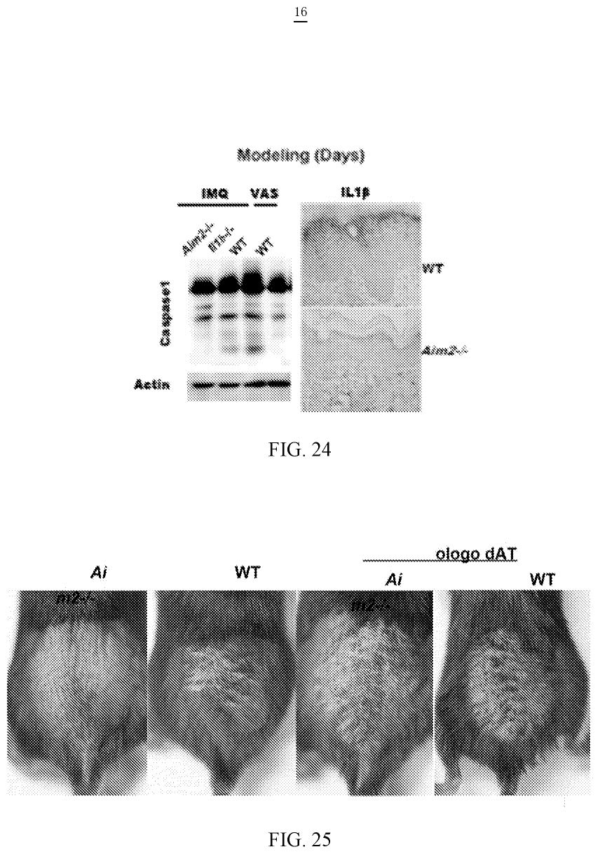

In this example of the invention, an IMQ mouse experiment and a dsDNA aggravation experiment after targeted knockout of the Aim2 and IL1b genes of the Aim2 pathway were performed. The results are shown in FIG. 22, FIG. 23, FIG. 24, and FIG. 25. The imq-induced psoriasis-like inflammatory response in Aim2 KO mouse was mild and recovered quickly (PASI and HE/tunel: dsDNA A. in cytoplasma B. higher level in ps skin lesion/western: aim2 pycard caspase1), indicating that the Aim2-mediated inflammasome pathway played an important role in the maintenance and deterioration of the psoriasis response.

### Example 13

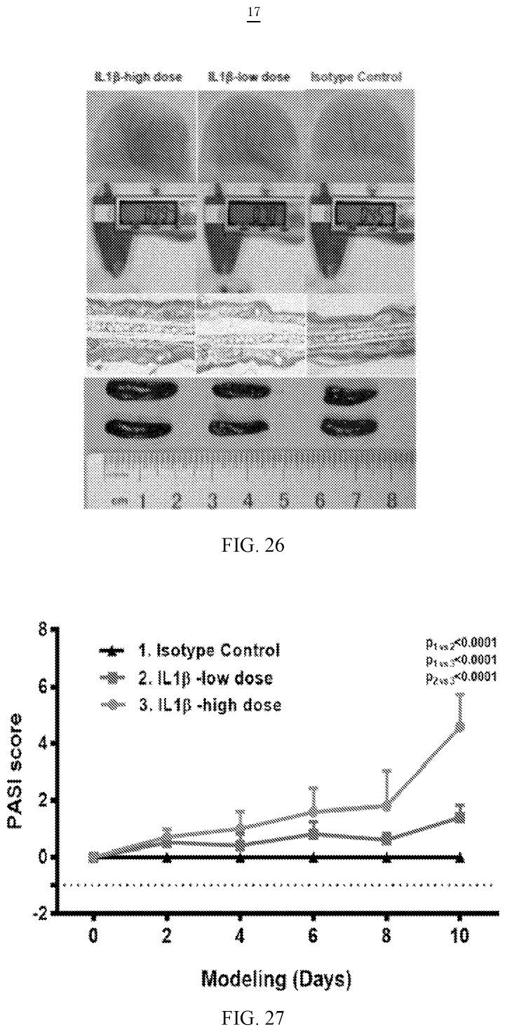

dsDNA-AIM2 finally released the cytokine IL-1b to act on the body. Clinical tests also showed that the skin tissue lesions and serum of psoriasis and the results of animal experiments showed that IL-1b was significantly increased in psoriasis. In order to identify whether the cytokine IL-1b alone can cause psoriasis-like response in a mouse, this example of the invention used IL-1b to inject ears of the mouse so as to cause psoriasis-like skin phenotype, spleen enlargement and pathological changes. The results are shown in FIG. 26 and FIG. 27.

### Example 14

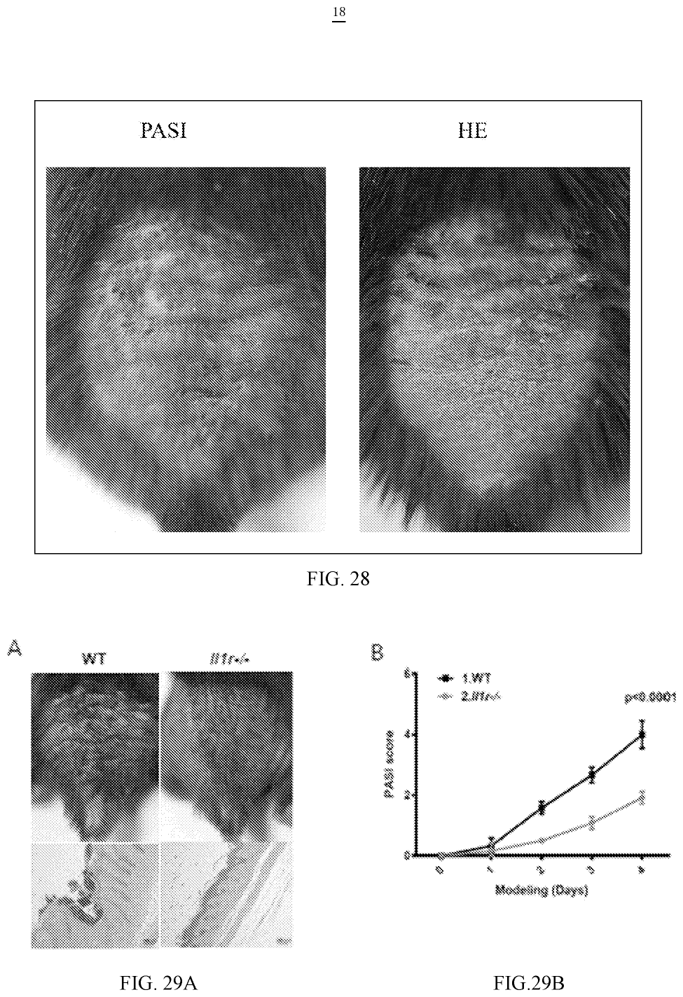

Because dsDNA-AIM2 finally released the cytokine IL-1b to act on the body to aggravate psoriasis, if IL-1b played an important role in imiquimoud-induced mouse psoriasis phenotype, the phenotype would be weakened after reduction of IL-1b activity. For this reason, this example of the invention used an IL-1b antibody to neutralize IL-1b biological activity. The experimental results are shown in FIG. 28. The results show that the 20× antibody can effectively alleviate the phenotype of the imq mouse model and achieve early recovery (PASI and HE).

### Example 15

In order to verify the important role of the signaling pathway of the cytokine IL1b released after activation of this pathway in the pathogenesis of psoriasis, this example of the invention knocked out the gene of mouse IL1b receptor IL1r, and again imiquimod was used for induction and the phenotype change was observed. The results in FIGS. 29A and 29B show that the phenotype after knockout of the gene of IL1b receptor IL1r is the same as that after AIM2 and IL1B KO, and the psoriasis phenotype is significantly alleviated.

### Example 16

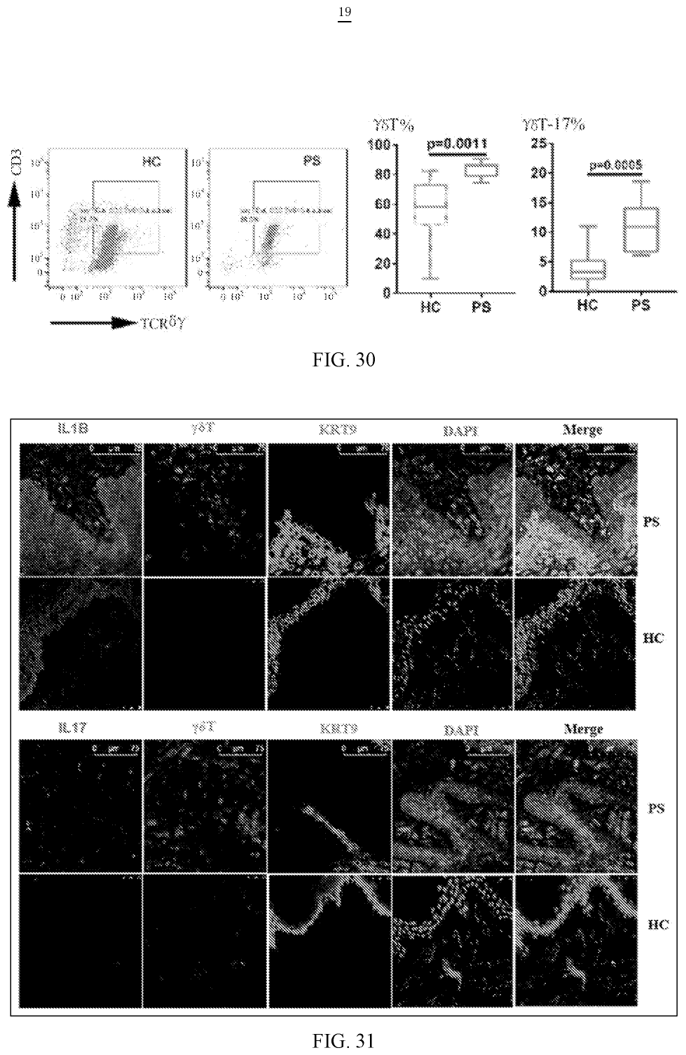

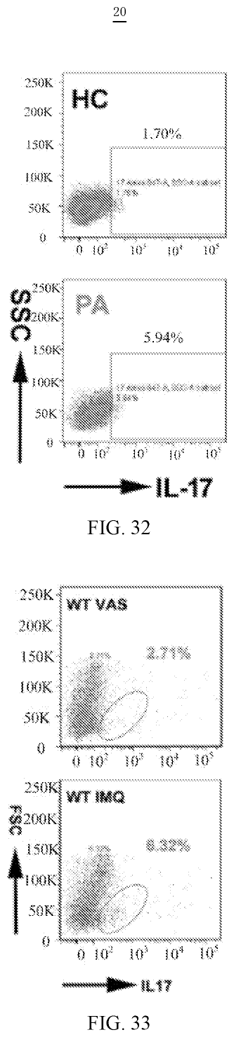

IL17 is an important pathogenic factor for psoriasis. Studies have shown that γδ T cells are an important source of skin IL17, and are the most relevant T cell for psoriasis. This example of the invention was to find the effect of dsDNA-AIM2-IL1b activation in the skin on the IL17 secretion by γδ T cells, and the IL17 secretion by γδ T cells was detected on the psoriasis-induced mouse model and after knockout of genes of AIM2, IL1b and IL1b receptor IL1r, respectively. The results in FIG. 30, FIG. 31, FIG. 32, and FIG. 33 show that the main source of IL17 in the skin is γδ T cells, and the IL17 secretion by γδ T cells is significantly reduced after the pathway is blocked.

### Example 17

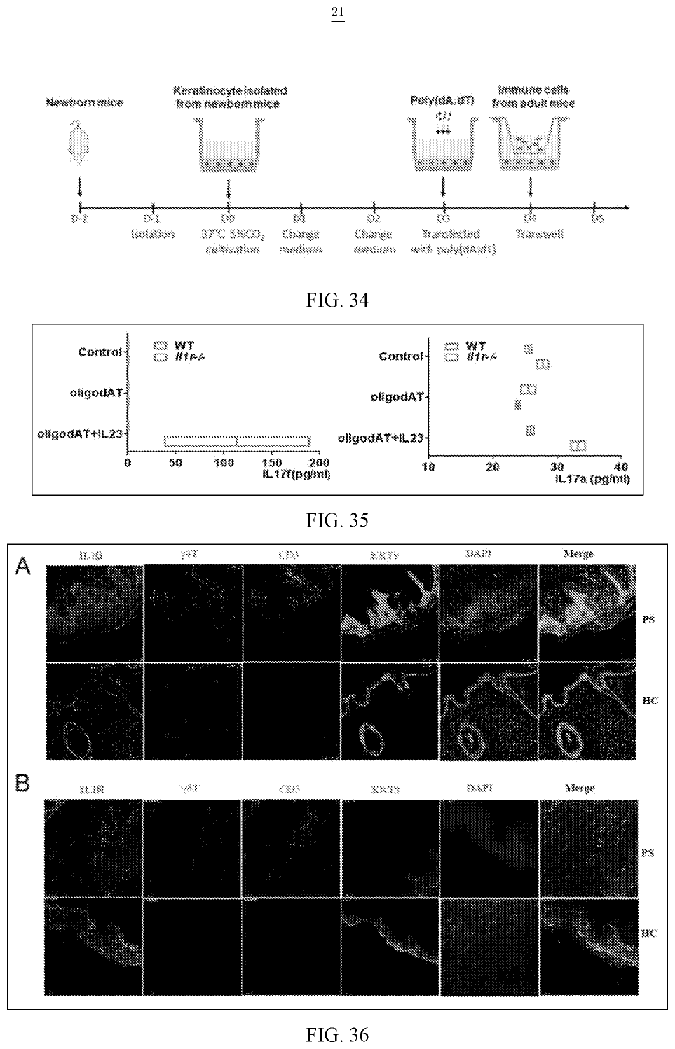

Transwell experiment showed that activation of the dsDNA-AIM2-ILIB pathway mainly affected the development of γδT-17. The mouse keratinocytes and immune cells were isolated for co-culture, and oligodAT and IL23 were added exogenously for in vitro stimulation (as shown in FIG. 34). After the experiment, the content of IL17 in the supernatant was detected. This example of the invention detected two cytokines A and F related to IL17 and psoriasis, respectively. The results are shown in FIG. 35. The results show that the IL1 signaling pathway is an essential factor for IL17A/F secretion and mainly stimulates the production of IL17F.

### Example 18

Keratinocytes in skin tissue are the main source of IL1b, and the IL1B receptor system of the keratinocyte system is inhibited during the occurrence of psoriasis, while the IL1B receptor system of the immune cell system is activated.

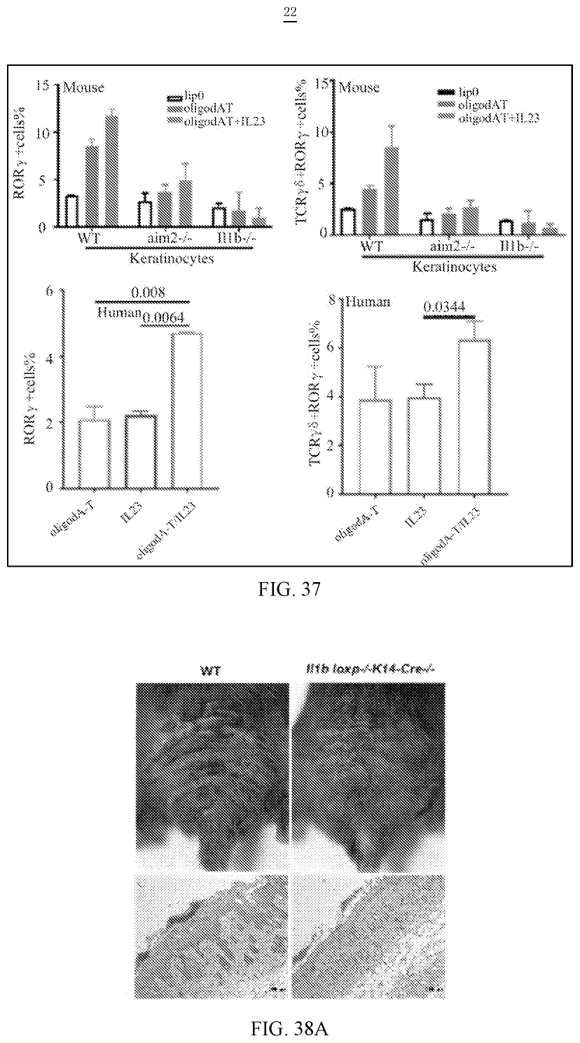

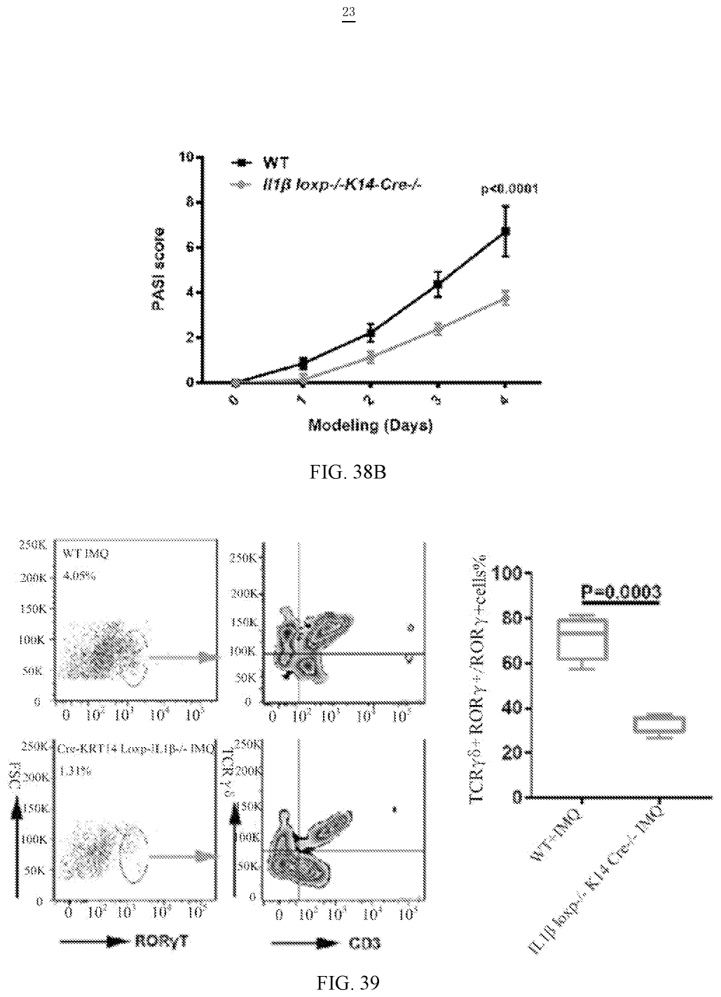

In Example 16, immunofluorescence co-labeling reveals that IL1b is mainly located in keratinocytes. In order to determine the role of keratinocyte-derived IL1b in the pathogenesis of psoriasis, in this example of the invention, relevant detections were carried out through in vitro experiments, and the results are shown in FIG. 36, FIG. 37, FIGS. 38A-38B, and FIG. 39. Immunofluorescence multi-labeling: the collected clinical skin samples were rinsed with PBS and then put into 30% sucrose solution and dehydrated in a refrigerator at 4° C. After the skin tissue sinked to the bottom of the fixed container, a freezing microtome was used to obtain a section with a thickness of 20 microns. Keratinocyte antibodies, lymphocyte antibodies and TCRγ antibodies with different fluorescent labels were used for multiple fluorescent staining, and finally DAPI was used for nuclear labeling. The slides were mounted with an anti-quenching agent and a laser confocal microscope was used for observation and photography. Interaction of keratinocytes and immune cells in vitro: the extracted human skin keratinocytes and mouse skin keratinocytes with different genotypes were cultured in the lower layer of the transwell chamber. Different stimuli were added. The isolated human primary skin lymphocytes and wild-type mouse skin lymphocytes were added to the upper layer. After co-culture, the supernatant and cells were collected for related detection.

### Example 19 Verification of γδT-17/RORγT Migration to the Epidermis

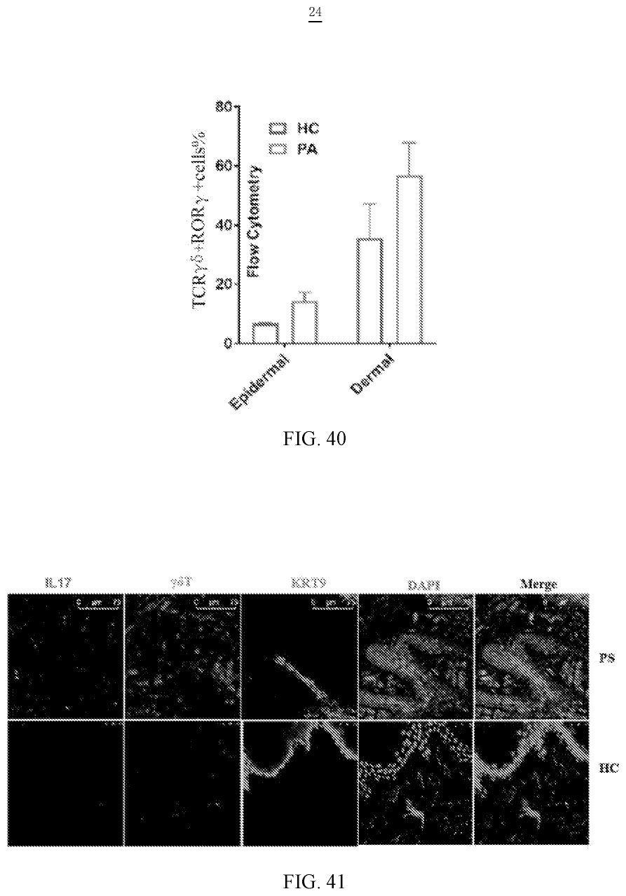

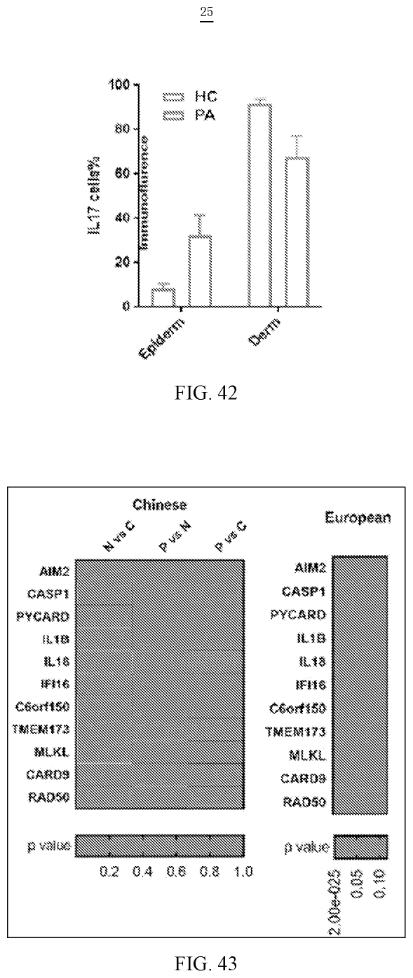

Flow cytometry was performed by isolating immune cells from the epidermis and dermis. It was found that γδT-17/RORγT in the epidermis of a patient with psoriasis and a mouse increased significantly (as shown in FIG. 40). The results of immunofluorescence detection also showed that positive cells in the epidermis increased (as shown in FIG. 41 and FIG. 42). These results show that the migration of γδT-17/RORγT to the epidermis is a pathological sign of psoriasis, would also enhance the effect of the dsDNA-AIM2 pathway in the keratinocytes, accelerate the release of cytokine IL1B, and accelerate the formation of circulatory effect of pathological dsDNA-AIM2-IL1B-IL17 in psoriasis.

### Example 20 Significant Correlation of dsDNA-AIM2 Pathway with Psoriasis at Multiple Levels

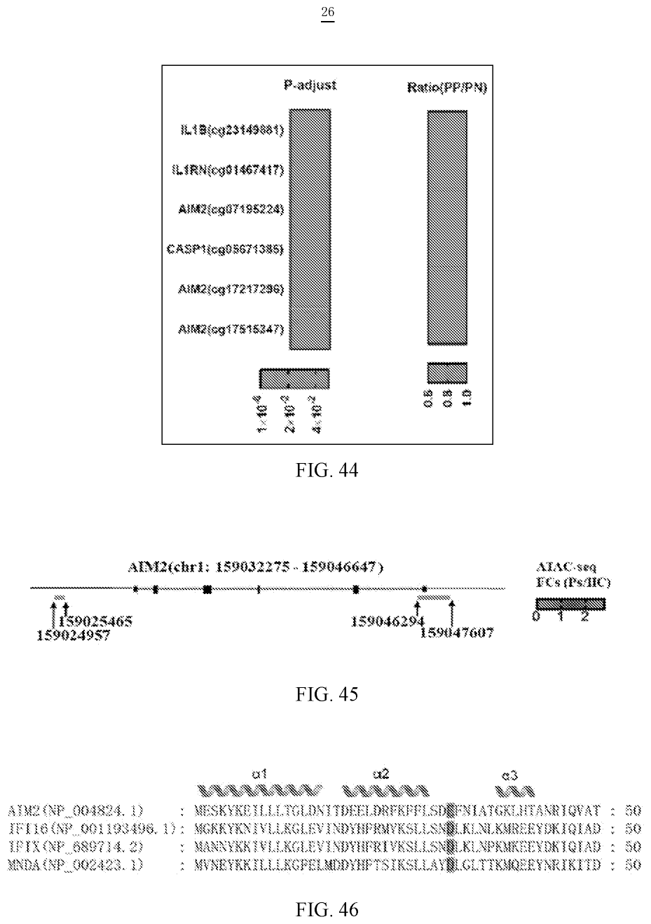

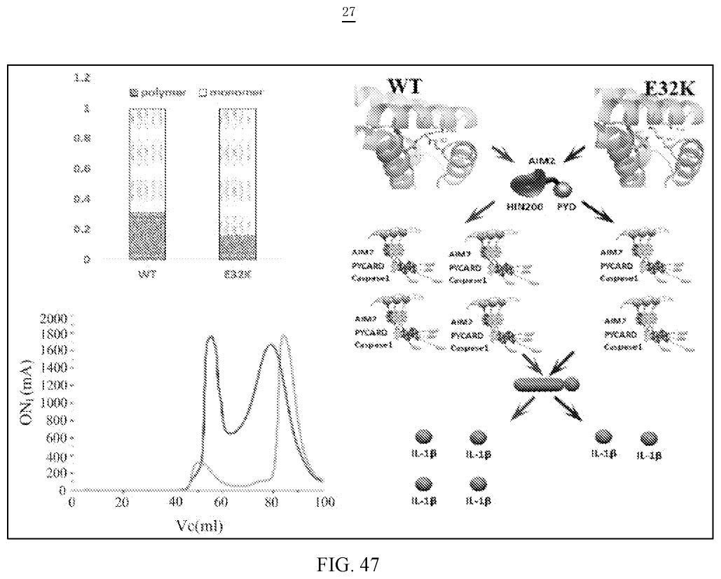

In this example of the invention, the dsDNA-AIM2 pathway was found to be significantly associated with psoriasis by means of transcriptome sequencing (as shown in FIG. 43), single-cell sequencing at transcription level, DNA methylation level detection (as shown in FIG. 44) and ATAC sequencing (as shown in FIG. 45). The inventors found that the conservative AIM2 E32K mutant has a protective effect against psoriasis through whole exome sequencing (as shown in FIG. 46).

Subsequently, through bioinformatics prediction and in vitro protein expression polymerization experiment, this example of the invention showed that the mutation of 32AA from an acidic amino acid to a basic amino acid affected the polymerization ability of AIM2 and weakened the active form of the polymer forming dsDNA-AIM2 (as shown in FIG. 47), that is, weakened the function of this pathway and reduced inflammation response.

Finally, it should be noted that the above examples are only used to illustrate the technical solutions of the invention and not to limit them. Although the invention has been described in detail with reference to the preferred examples, those of ordinary skill in the art should understand that the technical solutions of the invention can be modified or equivalently replaced without departing from the purpose and scope of the technical solutions of the invention, and all of them shall be covered by the scope of the claims of the invention.

What is claimed is:

1. An application method of an agent in preparation of a medicine for treating/inhibiting psoriasis, wherein the agent is at least one selected from the following agents (1) through (10):
(1) an interleukin 1b antagonist, and a biological activity inhibitor or functional analogue thereof;
(2) a double-stranded deoxyribonucleic acid (dsDNA) antagonist, and a biological activity inhibitor or functional analogue thereof or a dsDNA degrading agent;
(3) an absent-in-melanoma-2 (AIM2) antagonist, and a biological activity inhibitor or functional analogue thereof;
(4) a caspase1 antagonist, and a biological activity inhibitor or functional analogue thereof;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or functional analogue thereof;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.

2. The application method according to claim 1, wherein the agent is selected from IL-1β monoclonal antibodies.

3. The application method according to claim 1, wherein a dosage form of the medicine for treating/inhibiting psoriasis is a dressing, an oral medicament, a subcutaneous injection, or an intravenous injection.

4. An application method of an agent in preparation of an inhibitor for IL17 secretion by γδ T cells, wherein the agent is at least one selected from the following agents (1) through (10):
(1) an interleukin 1b antagonist, and a biological activity inhibitor or its functional analogue;
(2) a dsDNA antagonist, and a biological activity inhibitor or its functional analogue or a dsDNA degrading agent;
(3) an AIM2 antagonist, and a biological activity inhibitor or its functional analogue;
(4) a caspase1 antagonist, and a biological activity inhibitor or its functional analogue;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or its functional analogue;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.

5. An application method of an agent in preparation of a medicament for inhibiting spread of skin tissue lesions, wherein the agent is at least one selected from the following agents (1) through (10):
(1) an interleukin 1b antagonist, and a biological activity inhibitor or its functional analogue;
(2) a dsDNA antagonist, and a biological activity inhibitor or its functional analogue or a dsDNA degrading agent;
(3) an AIM2 antagonist, and a biological activity inhibitor or its functional analogue;
(4) a caspase1 antagonist, and a biological activity inhibitor or its functional analogue;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or its functional analogue;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.

6. An application method of an agent in preparation of an inhibitor for keratinocyte hyperproliferation in skin tissue, wherein the agent is at least one selected from the following agents (1) through (10):
(1) an interleukin 1b antagonist, and a biological activity inhibitor or its functional analogue;
(2) a dsDNA antagonist, and a biological activity inhibitor or its functional analogue or a dsDNA degrading agent;
(3) an AIM2 antagonist, and a biological activity inhibitor or its functional analogue;
(4) a caspase1 antagonist, and a biological activity inhibitor or its functional analogue;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or its functional analogue;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.

7. An application method of an agent in preparation of an inhibitor for AIM2 inflammasome response, wherein the agent is at least one selected from the following agents (1) through (10):
(1) an interleukin 1b antagonist, and a biological activity inhibitor or its functional analogue;
(2) a dsDNA antagonist, and a biological activity inhibitor or its functional analogue or a dsDNA degrading agent;
(3) an AIM2 antagonist, and a biological activity inhibitor or its functional analogue;
(4) a caspase1 antagonist, and a biological activity inhibitor or its functional analogue;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or its functional analogue;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.

8. A method for inhibiting IL17 secretion by γδ T cells in vitro, wherein the method comprises knocking out one or more selected from the group consisting of an AIM2-expressing gene, an IL1b-expressing gene, and a receptor IL1r-expressing gene in cells ex vivo.

9. A method for inhibiting AIM2 inflammasome response in vitro, wherein the method comprises using at least one of the following agents (1) through (10) for interference:
(1) an interleukin 1b antagonist, and a biological activity inhibitor or functional analogue thereof;
(2) a dsDNA antagonist, and a biological activity inhibitor or functional analogue thereof or a dsDNA degrading agent;
(3) an AIM2 antagonist, and a biological activity inhibitor or functional analogue thereof;
(4) a caspase1 antagonist, and a biological activity inhibitor or functional analogue thereof;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or functional analogue thereof;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.

10. A method for inhibiting keratinocyte hyperproliferation in skin tissue in vitro, wherein the method comprises using at least one of the following agents (1) through (10) for interference:
(1) an interleukin 1b antagonist, and a biological activity inhibitor or its functional analogue;
(2) a dsDNA antagonist, and a biological activity inhibitor or its functional analogue or a dsDNA degrading agent;
(3) an AIM2 antagonist, and a biological activity inhibitor or its functional analogue;
(4) a caspase1 antagonist, and a biological activity inhibitor or its functional analogue;
(5) an interleukin 1b expression inhibitor, a dsDNA production inhibitor, and an AIM2 expression inhibitor or a caspase1 expression inhibitor;
(6) an AIM2 polymerization inhibitor, or a dsDNA binding agent or its functional analogue;
(7) an AIM2 gene DNA methylation promoting agent;
(8) an interleukin 1r1 binding agent, or an interleukin 1r2 binding agent;
(9) an interleukin 1ra expression promoting agent; and
(10) an interleukin 17a expression promoting agent, or an interleukin 17f expression promoting agent.
